(19) United States
(12) Patent Application Publication
(10) Pub. No.: US 2026/0024660 A1
(43) Pub. Date: Jan. 22, 2026

(54) GLAUCOMA DETERMINATION SYSTEM AND GLAUCOMA DETERMINATION METHOD

(71) Applicants: Tohoku University, Sendai-shi, Miyagi (JP); CASIO COMPUTER CO., LTD., Tokyo (JP); ROHTO Pharmaceutical Co., Ltd., Osaka-shi, Osaka (JP)

(72) Inventors: Toru Nakazawa, Sendai-shi, Miyagi (JP); Takahiro Tomida, Hamura-shi, Tokyo (JP); Toshihiko OTSUKA, Hamura-shi, Tokyo (JP); Harumasa Arita, Osaka-shi, Osaka (JP)

(21) Appl. No.: 19/107,517

(22) PCT Filed: Aug. 21, 2023
(86) PCT No.: PCT/JP2023/030029

§ 371 Date: Feb. 27, 2025

(30) Foreign Application Priority Data
Aug. 31, 2022 (JP) 2022-138232

(51) Int. Cl. G16H 50/20; A61B 3/12; A61B 3/14; A61B 5/021

(52) U.S. Cl. CPC G16H 50/20; A61B 3/1241; A61B 3/14; A61B 5/02116; A61B 5/02125

(57) ABSTRACT

There is provided a system that determines the possibility of glaucoma without a large-scale measurement of a myoelectric potential of the heart. A glaucoma determination system (1) includes a processor (112) that acquires index data related to blood flow of a subject, determines, based on the acquired index data, the possibility of glaucoma in an eye of the subject, and outputs information related to the determined possibility of glaucoma.

### TECHNICAL FIELD

The present invention relates to a system for diagnostic support and prediction of glaucoma or pre-symptomatic glaucoma by estimating a change in retinal blood flow from a change in blood flow pertaining to an autonomic reflex of a subject.

### BACKGROUND ART

Glaucoma, which is said to affect 1 in 20 Japanese people aged 40 years or older, is a serious retinal disease that is one of the leading causes of blindness. Glaucoma is a disease causing a gradual defect of the visual field due to a damage of the optic nerve caused by some reason, and in the early stages of glaucoma where a very slight defect of the visual field is involved, patients usually notice no or very few subjective symptoms. This poses a problem that it is difficult for glaucoma patients in the early stages to notice the disease through subjective symptoms.

Glaucoma is classified into primary angle closure glaucoma and primary open angle glaucoma. The primary open angle glaucoma is further classified into primary open angle glaucoma in a narrow definition, in which intraocular pressure increases, and normal tension glaucoma in which no increase in intraocular pressure is observed, and this normal tension glaucoma is more common in Japanese people (about 90%). In the glaucoma involving an increase in intraocular pressure, a definitive diagnosis can be made based on the intraocular pressure that can simply be measured. However, in the case of the normal tension glaucoma, which accounts for the majority of glaucoma patients, there is a problem that the diagnosis cannot be made by measurement of the intraocular pressure and can only be made based on whether or not there is an actual visual field defect. There is also another problem that people are less motivated to get a visual field examination since there is few subjective symptoms of the visual field defect in the early stages in the first place.

However, in a pre-symptomatic state, there is often no signs (objective or subjective symptoms) indicating that the pre-symptomatic state is developing to the disease state in the first place. In addition, even if there is a sign indicating the risk before actually getting glaucoma, patients have very little motivation to consume time and cost for medical examination to ascertain the risk, at the stage where the patients are asymptomatic. Therefore, even if a system that can calculate (predict) the risk before actually getting the disease is developed, it is extremely difficult to put the system in practice in society to support sustainable health maintenance activities.

Visual field testing, which is a primary diagnostic method for normal tension glaucoma, is a method of determining the limit of visibility of a light index around the retina, the light index being set to appear in various positions of the visual field and to be gradually darkened. Because of its measurement principle, visual field testing is a very delicate measurement method that takes time (about 10 to 20 minutes), makes it difficult to maintain concentration, and is often hard to get correct measurement results due to sleepiness or other causes. Even when it is better to worry about glaucoma in consideration of age, or even when examination of the visual field is recommended at a medical checkup, the hurdle to take a visual field examination is quite high.

As noted above, even though glaucoma is a serious condition that can lead to blindness if not detected at early stages, its fatal challenge is that there is no easy way to diagnose glaucoma at a pre-symptomatic stage (before visual field defect) or at an early symptom (slight visual field defect) and that the normal tension glaucoma is only discovered after a significant range of the visual field is defected.

On the other hand, glaucoma is said to be correlated with some abnormalities in the autonomic nervous system, and cardiovascular autonomic nervous function and glaucoma have some correlation (Non Patent Literature 1). According to these studies, when electrocardiogram (ECG) data from patients suffering from normal tension glaucoma is compared with ECG data from patients suffering from primary open angle glaucoma in a narrow definition, there was a significant difference in specific ECG parameters. This suggests the possibility that measuring a myoelectric potential of the heart can assess normality of the autonomic nervous function and can diagnose the normal tension glaucoma, though it is not suggested nor mentioned that pre-symptomatic normal tension glaucoma or initial symptoms of normal tension glaucoma can be diagnosed.

### CITATION LIST

### Non Patent Literature

Non Patent Literature 1: “Assessment of parasympathetic cardiovascular activity in primary open-angle glaucoma” by Oluwaseun O. Awe, Ouwadare Ogundare, Bernice O. Adegbehingbe, published on Nov. 5, 2021, Int Ophthamol (2022) 42: p. 1111-1119

### SUMMARY OF INVENTION

### Technical Problem

The present disclosure is to determine the possibility of glaucoma in accordance with blood flow without a large-scale measurement of a myoelectric potential of the heart.

### Solution to Problem

In order to accomplish the above object, a glaucoma determination system according to one aspect of the present disclosure includes a processor that acquires index data related to blood flow of a subject, determines, based on the acquired index data, a possibility of glaucoma in an eye of the subject, and outputs information related to the possibility of the determined glaucoma.

In order to accomplish the above object, a glaucoma determination method executed by a computer according to another aspect of the present disclosure includes the processing steps of: acquiring index data related to blood flow of a subject; determining, based on the index data related to the blood flow of the subject, a possibility of glaucoma in an eye of the subject; and outputting information related to the possibility of the glaucoma.

In order to accomplish the above object, a program according to yet another aspect of the present disclosure causes a computer to execute: acquiring index data related to blood flow of a subject; determining, based on the index data related to the blood flow of the subject, a possibility of glaucoma in an eye of the subject; and outputting information related to the possibility of the glaucoma.

### Effects

According to the present disclosure, it is possible to determine the possibility of glaucoma in accordance with blood flow without a large-scale measurement of a myoelectric potential of the heart.

### BRIEF DESCRIPTION OF DRAWINGS

FIG. 1 is a functional block diagram of a system in a first embodiment.

FIG. 2 is a functional block diagram of the system in a second embodiment.

FIG. 3 is a functional block diagram of the system in a third embodiment.

FIG. 4 is a determination flow based on a change rate in pulse wave amplitude (APA).

FIG. 5 is a determination flow based on the change rate in pulse wave amplitude (APA) and a change rate in heart rate adjustment index (ACVRR).

FIG. 6 is a diagram showing an example of the change rate in pulse wave amplitude (APA) in a healthy group and an NTG group.

FIG. 7 is a diagram showing an example of the change rate in heart rate adjustment index (ACVRR) in the healthy group and the NTG group.

FIG. 8 is a diagram showing an example of the change rate in pulse wave amplitude (APA) in the healthy group and the NTG group using a coolant in another embodiment.

### DESCRIPTION OF EMBODIMENTS

### First Embodiment

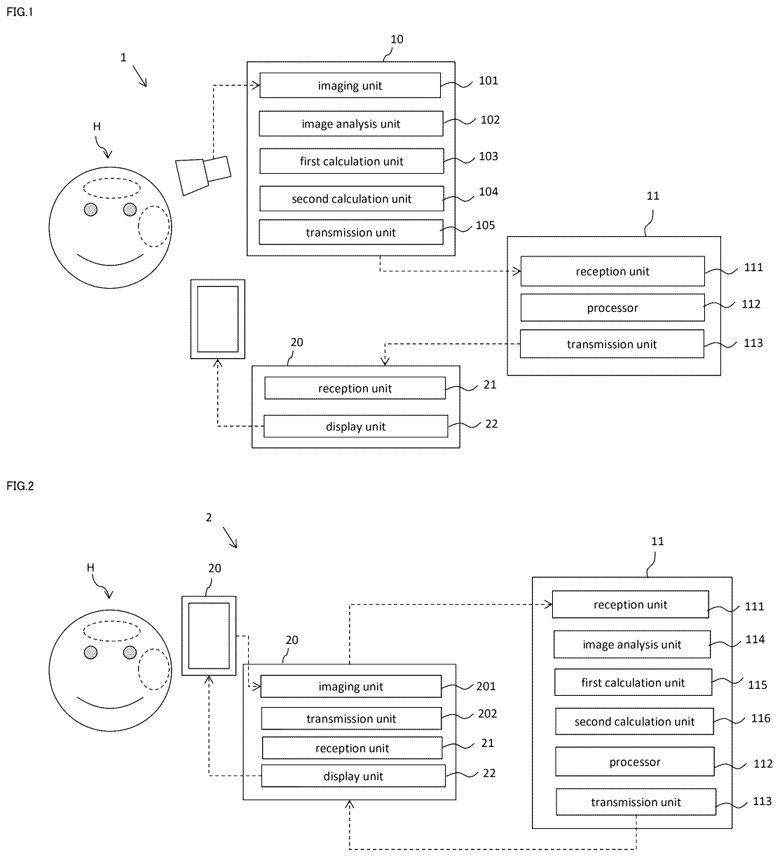

A glaucoma determination system 1 (hereinafter referred to as “system 1”) of the first embodiment is described with reference to FIG. 1.

The system 1 includes an imaging analyzer 10 that images a specific region of a subject H and performs image analysis to obtain an index related to blood flow, an information processor 11 having a processor 112, and a mobile terminal 20 that receives and displays the result of determination made in the processor 112.

The glaucoma determination system 1 is mainly used to assist determination, and this also applies to other embodiments as is described later.

The blood flow of the subject H may be the blood flow at a specific region. The specific region is not particularly limited and may be the face, neck, extremities, or torso, as long as blood flow can be measured. In consideration of easiness in measurement, the specific region may also be the forehead, cheek, palm, back of the hand, wrist, neck, ankle, or the like. The blood flow of the subject may be the blood flow after a prescribed load is applied to the subject. The “prescribed load” may be a load on the body or a load on a specific region. The “prescribed load” may also be heat provocation (cold provocation or heating provocation) on a specific region, a load caused by skin massage, a load caused by pressure, a load caused by pain stimulation, and event burden, such as a load caused by light exercise, and a load caused by washing of a specific region. A change rate in pulse wave amplitude (APA) and a fluctuation of pulse wave interval (for example, a change rate in heart rate adjustment index (ACVRR)) may be calculated from measurement values (averages) before and after the event burden.

In the first and second embodiments, the blood flow of the subject H is the blood flow obtained from a moving image of the skin of the subject. In a method of obtaining the blood flow from a moving image of the skin, a luminance value of a green signal (green color light) is acquired from the moving image. A pulse wave of blood flow may be obtained from a moving image using the method described in Japanese Patent Laid-Open No. 2022-52191, and a pulse wave amplitude and a pulse wave interval may be calculated.

In a third embodiment, which is described later, the blood flow of the subject H is the blood flow obtained from a light signal by transmitted light or reflected light. Examples of the means for measuring the blood flow (pulse wave) of the skin may include a transmissive pulse wave sensor, a reflective pulse wave sensor, or means for imaging the skin and measuring the pulse wave from a specific color (green).

The imaging analyzer 10 includes an imaging unit 101 that takes a moving image, an image analysis unit 102 that analyzes the blood flow from the moving image to obtain a pulse wave amplitude, a first calculation unit 103 that calculates a change rate in pulse wave amplitude (APA), a second calculation unit 104 that calculates fluctuation of a pulse wave interval (change rate in heart rate adjustment index (ACVRR)), and a transmission unit 105 that transmits each data obtained by each calculation function to the information processor 11.

The imaging unit 101 is implemented by, for example, a color camera, a CCD or CMOS image sensor. The image analysis unit 102 and the first and second calculation units 103, 104 are implemented by dedicated circuits, hardware such as processors that execute programs including processing procedures, or the like. The transmission unit 105 is implemented by wireless communication means such as Wi-Fi and Bluetooth (registered trademark), wired communication means, or the like. The functions of the image analysis unit 102 and the first and second calculation units 103, 104 are described later.

The information processor 11, which includes a reception unit 111, a processor 112, and a transmission unit 113, is implemented by a general-purpose computer, a local server, and a cloud server. The reception unit 111 is implemented by reception means for receiving data from the imaging analyzer 10. The transmission unit 113 is constituted of transmission means for sending the result of determination made in the processor 112 to the mobile terminal 20. The transmission means and the reception means are implemented by means of, for example, wireless communication such as Wi-Fi and Bluetooth (registered trademark) and wired communication. The functions of the processor 112 are described later.

The mobile terminal 20 includes a reception unit 21 that receives the result of determination made in the processor 112 and a display unit 22 that displays the result on a screen. The mobile terminal 20 may have a glaucoma detection application installed thereon and be configured to display the determination result upon start of the application. The transmission unit 113 can also send data in an e-mail format or a short message format to the mobile terminal 20. In this case, the mobile terminal 20 may be configured to receive an e-mail or a short message with a mailer function or a short message function. The reception unit 21 is implemented by wireless communication means, such as Wi-Fi and Bluetooth (registered trademark). The display unit 22 may be constituted of, for example, an output interface such as a liquid crystal panel or an organic EL panel.

### Second Embodiment

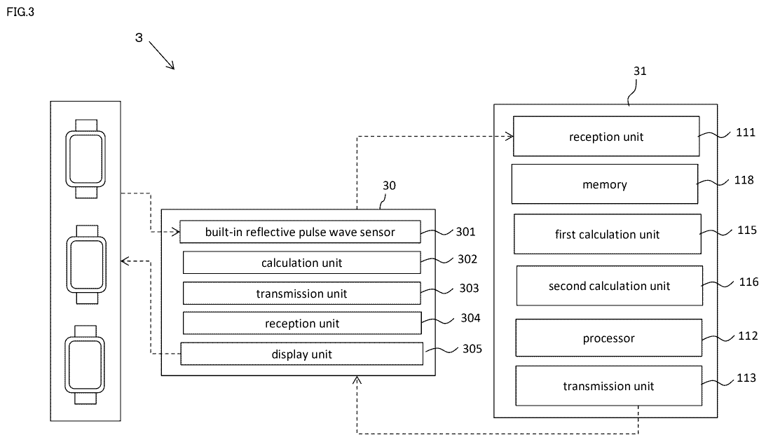

A glaucoma determination system 2 (hereinafter referred to as “system 2”) of a second embodiment is described with reference to FIG. 2.

The system 2 includes a mobile terminal 20 including an imaging unit 201 that images a specific region of the subject H, and an information processor 11 including an image analysis unit 114, a first calculation unit 115, a second calculation unit 116, and a processor 112.

The imaging unit 201 of the mobile terminal 20 is implemented by, for example, a color camera, a CCD or CMOS image sensor. The mobile terminal 20 includes a transmission unit 202 that sends a taken moving image to the information processor 11, a reception unit 21 that receives the result of determination made in the processing unit 112, and a display unit 22 that displays the result on the screen. The transmission unit 202 and the reception unit 21 are implemented by, for example, wireless communication means such as Wi-Fi and Bluetooth (registered trademark). The display unit 22 is implemented by the same means as described above.

The information processor 11 is implemented by a general-purpose computer, a local server, and a cloud server. The reception unit 111 is implemented by reception means for receiving data from the mobile terminal 20. The transmission unit 113 is constituted of transmission means for sending the result of determination made in the processor 112 to the mobile terminal 20. The reception means and transmission means are implemented by the same means as described above.

Description is given of the image analysis unit 114. The image analysis unit 102 in the first embodiment also has the same configuration.

The image analysis unit 114 acquires a luminance value of a green signal (green light) from the moving image, obtains a pulse wave of the blood flow of the subject, and calculates a pulse wave amplitude and a pulse wave interval over time. As a method of calculating the pulse wave amplitude and the pulse wave interval in this case, the method disclosed in Japanese Patent Laid-Open No. 2022-52191 can be used.

Description is given of the first calculation unit 115. The first calculation unit 103 in the first embodiment also has the same configuration.

The first calculation unit 115 calculates from the blood flow of the subject a change rate in pulse wave amplitude (APA) that is a change rate in pulse wave amplitude (PA) at a prescribed time. Specifically, the pulse amplitude change rate (APA) is calculated by a following expression (1):

$$Pulse wave amplitude change rate ( Δ PA ) = ( PA 2 / PA 1 ) - 1 \qquad ( 1 )$$

where
PA1 is an average of pulse wave amplitude measurement values over n seconds in a first measurement,
PA2 is an average of pulse wave amplitude measurement values over n seconds in a second measurement,
n may be set to, for example, a duration of time (second) in which two or more pulses are included.
the first measurement is the measurement before an event (including load on the skin),
the second measurement is the measurement during or after the event (including load on the skin), and
the pulse amplitude is a peak of the waveform of a measured pulse wave.

The first measurement is desirably performed at the timing just a prescribed time (for example, a few seconds) before a time point when a prescribed load is applied. In this way, the first measurement is performed at the timing a prescribed time before the time point of applying a prescribed load, that is, at the timing just before the prescribed load is applied, in order to prevent that the pulse amplitude fluctuates between the first measurement and the second measurement due to the reason that stimulation other than the event or the like affects the pulse amplitude of the subject H after the first measurement. The measurement is also desirably performed just before application of the prescribed load. Since there is a time interval between the first measurement and the second measurement, various changes in mind and body may appear and therefore, the state just before application of the load is measured as a base value (PA1) before change. The second measurement is performed at the timing of, for example, the lapse of 2 minutes, 4 minutes, or 6 minutes from a time point when application of the prescribed load is completed.

The change rate in pulse wave amplitude (ΔPA) may also be calculated based on the average pulse wave amplitude over a prescribed period. In the case of calculating the change rate in pulse wave amplitude (ΔPA) based on an average of the measurement values measured over the prescribed period, the change rate in pulse wave amplitude (ΔPA) is calculated by a following expression (2):

$$Pulse amplitude change rate ( Δ PA ) = ( PAt / PAaverage ) - 1 , \qquad ( 2 )$$

where
PAaverage is an average of measurement values measured over a prescribed period;
PAt is an average of measurement values of pulse wave amplitude over n seconds in the measurement performed at an optional timing (time t), and
n may be set to, for example, a duration of time (second) in which two or more pulses are included.

The “prescribed period” of PAaverage may be an interval in minutes such as 1 to 10 minutes, an interval in hours such as 1 to 3 hours, an interval in days such as 1 to 3 days, an interval in weeks such as 1 to 3 weeks, an interval in months such as 1 to 3 months, or an interval in years such as 1 to 3 years. The “prescribed interval” may be set in accordance with the conditions of use of the device for measuring or the device for calculating the index data related to the blood flow, or the configuration of the system. Note that PAaverage is stored in a system memory (not shown).

The optional timing is set according to the purpose of the measurement. Examples of the optional timing includes after the load, after meal, after exercise, after overuse of the eyes, and the timing of a preliminary measurement in a medical checkup.

The optional timing PAt may also be, for example, the timing after the lapse of 2 minutes, 4 minutes, or 6 minutes from a time point when the subject returns to a room from the outside, with a pulse wave amplitude of the subject when the subject is inside the room being used as PAaverage. In this case, the system may be configured to start measurement related to blood flow upon the lapse of a prescribed time from a time point when an external temperature measured by a temperature sensor that is built into a wearable terminal is equal to or more than the prescribed temperature, so that the measurement can be started automatically.

Description is given of the second calculation unit 116. The second calculation unit 104 in the first embodiment also has the same configuration.

The second calculation unit 116 calculates fluctuation of the pulse wave interval based on the blood flow. In the present embodiment, the second calculation unit 116 calculates as the fluctuation of the pulse wave interval a change rate in heart rate adjustment index (ΔCVRR) that is a change rate in a heart rate adjustment index (CVRR) over a prescribed time. First, the heart rate adjustment index (CVRR) is calculated by a following expression (3). The heart rate adjustment index (CVRR) is calculated in the second calculation unit 116 or in the image analysis unit.

$$Heart rate adjustment index ( CVRR ) = standard deviation \\ of pulse wave variation / average pulse wave interval \times 100 \qquad ( 3 )$$

The average pulse wave interval is an average of peak intervals of the waveform of continuously measured pulse wave.

The standard deviation of the pulse wave variation is a standard deviation of the peak intervals of the waveform of continuously measured pulse wave.

A measurement time of at least 60 seconds is required to calculate the heart rate adjustment index (CVRR). The “measurement time” may be set in accordance with the conditions of use of the device for measuring or the device for calculating the index data related to the autonomic nervous system, or the configuration of the system.

Next, the change rate in heart rate adjustment index (ΔCVRR) is calculated by a following expression (4):

$$Change rate in heart rate adjustment index ( Δ CVRR ) = ( CVRR 2 / CVRR 1 ) - 1 , \qquad ( 4 )$$

where
CVRR1 is a value in the first measurement, the value being measured over 60 seconds or more and calculated by the expression (3),
CVRR2 is a value in the second measurement, the value being measured over 60 seconds or more and calculated by the expression (3),
the first measurement is the measurement before an event (including a load on the skin), and
the second measurement is the measurement during or after the event (including a load on the skin).

For the first measurement, an average of the heart rate adjustment index values (CVRRaverage) over a long period of time may be adopted. The average CVRRaverage may be an average of a plurality of the heart rate adjustment index values (CVRR) intermittently measured and calculated over any period, for example, one hour or more, one day or more, one week or more, or one month or more. The heart rate adjustment index CVRRt in the second measurement may be measured at an optional timing (time t).

The first and second measurements may be performed at the same timing as that of the change rate in pulse wave amplitude described above.

The measurement timing of the average CVRRaverage and the optional timing CVRRt may be the same as an average of the change rate in pulse wave amplitude and the optional timing.

Description is given of the processor 112. The processor 112 determines, based on the index data related to the blood flow of the subject H, a possibility and a degree of glaucoma in an eye of the subject H, and outputs information related to the possibility and the degree of the glaucoma.

The “information related to the possibility of glaucoma” may be, for example, being healthy (no possibility of glaucoma), having the possibility of glaucoma, observation required due to the possibility of glaucoma, re-examination required a prescribed month (for example, 3 or 6 months) later, further examination required, or other information.

The “degree of glaucoma” may be divided into a plurality of ranks, such as mild glaucoma suspected, moderate glaucoma suspected, or severe glaucoma suspected.

In the first and second embodiments, the processor 112 determines the possibility of glaucoma and the degree of glaucoma based on the results of comparison between the change rate in pulse wave amplitude (ΔPA) and a prescribed threshold and between the change rate in heart rate adjustment index (ΔCVRR) and a prescribed threshold. The “prescribed threshold” may be set in advance and may be changed in accordance with the use status of the system, and mental and physical conditions of the subject. Thresholds may be set using average data pertaining to healthy individuals, a pre-glaucoma group, a pseudo-glaucoma group, and a glaucoma patient group.

The processor 112 determines the possibility and the degree of glaucoma based on, for example, a plurality of following thresholds.
(A) Determination based on change rate in pulse wave amplitude (ΔPA).
(A1) Pulse wave amplitude change rate (ΔPA) is less than a first threshold (Th₁): healthy group
(A2) Pulse wave amplitude change rate (ΔPA) is equal to or more than the first threshold (Th₁) and less than a second threshold (Th₂): pre-glaucoma group

The pre-glaucoma group is, for example, a pre-symptomatic group that requires observation and re-examination.

(A3) Pulse wave amplitude change rate (ΔPA) is equal to or more than the second threshold (Th₂) and less than a third threshold (Th₃): pseudo-glaucoma group

The pseudo-glaucoma group is, for example, a pre-symptomatic or unaware group that requires further examination (visual field examination in hospital).

(A4) Pulse wave amplitude change rate (ΔPA) is equal to or more than the third threshold (Th₃): glaucoma group

More thresholds may be set to divide the glaucoma group into mild, moderate, severe, and the like. The size relation of each threshold is first threshold (Th₁)>second threshold (Th₂)>third threshold (Th₃).

(B) Determination based on change rate in heart rate adjustment index (ΔCVRR)

(B4) Change rate in heart rate adjustment index (ΔCVRR) is less than a fifth threshold (Th₅): glaucoma group

(B3) Change rate in heart rate adjustment index (ΔCVRR) is equal to or more than a fifth threshold (Th₅) and less than a sixth threshold (Th₂): pseudo-glaucoma group

(B2) Change rate in heart rate adjustment index (ΔCVRR) is equal to or more than the sixth threshold (Th₆) and less than a seventh threshold (Th₇): pre-glaucoma group

(B1) Change rate in heart rate adjustment index (ΔCVRR) is equal to or more than the seventh threshold (Th₇): healthy group

The size relation of each threshold is seventh threshold (Th₇)>sixth threshold (Th₆)>fifth threshold (Th₅).

(C) Determination based on change rate in pulse wave amplitude (ΔPA) and change rate in heart rate adjustment index (ΔCVRR)

(C1) When both A1 and B4 are satisfied: healthy group

(C2) When both A2 and B3 are satisfied: pre-glaucoma group

(C3) When both A3 and B2 are satisfied: pseudo-glaucoma group

(C4) When both A4 and B1 are satisfied: glaucoma group

The processor 112 may set each threshold by using, for example, two types of average data on healthy individuals and the glaucoma patient group. In this case, a middle value between a threshold based on the average data on the healthy individuals and a threshold based on the average data on the glaucoma patient group may be set as a threshold for (pre-glaucoma group and the pseudo-glaucoma group). When setting the threshold, the system may be configured to compensate the pre-set thresholds in consideration of individual differences.

### Third Embodiment

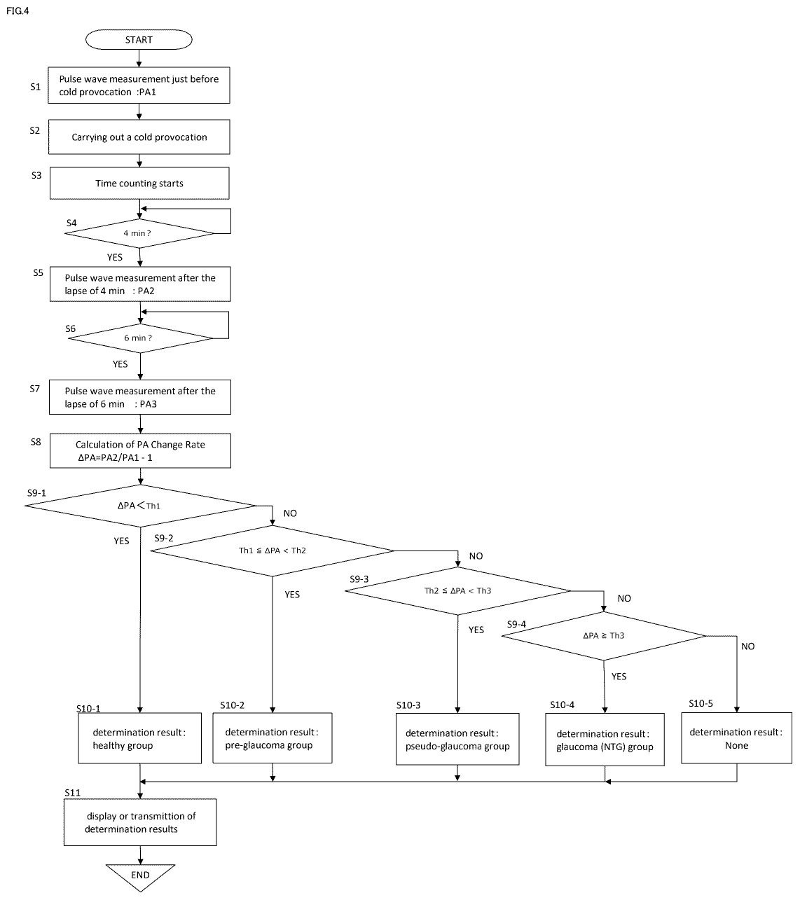

A glaucoma determination system 3 (hereinafter referred to as “system 3”) of a third embodiment is described with reference to FIG. 3.

The system 3 includes a plurality of wearable terminals 30 of a smartwatch-type that is attached to the list of the subject H or a neck-hanging type, an image analysis unit 114, a first calculation unit 115, a second calculation unit 116, and a cloud server 31 having a processor 112.

The wearable terminal 30, which has a built-in reflective pulse wave sensor 301, irradiates blood vessels with light from the sensor 301 and receives the reflected light from the blood vessels. The wearable terminal 30 includes a calculation unit 302 that analyzes the pulse wave from the received reflected light and calculates the pulse wave amplitude and pulse wave interval. In another embodiment, when reflected light data is sent to the cloud server 31, the cloud server 31 may include the function of the calculation unit. The wearable terminal 30 includes a transmission unit 303 that transmits each data of the pulse wave amplitude and pulse wave interval to the cloud server 31. The wearable terminal 30 may include a reception unit 304 that receives the result of determination made in the processor 112, and a display unit 305 that displays the received determination result. The transmission unit 303 and the reception unit 304 are implemented by the aforementioned wireless communication means or the like. The display unit 305 is implemented by the same means as described above.

The cloud server 31 receives the pulse wave amplitude and pulse wave interval from each wearable terminal 30 through the reception means of the reception unit 111. During acquisition of the pulse wave amplitude and pulse wave interval, the cloud server 31 also acquires identification information of the wearable terminals 30, and stores in a memory 118 the pulse wave amplitude and the pulse wave interval for each wearable terminal 30. The functions of the first calculation unit 115, the second calculation unit 116, and the processor 112 are as described in the second embodiment. The difference is that the change rate in pulse wave amplitude (ΔPA) and the change rate in heart rate adjustment index (ΔCVRR) are calculated for each of the wearable terminals 30, and determination regarding glaucoma is executed.

The transmission unit 113 transmits to each of the wearable terminals 30 the determination result corresponding to each of the wearable terminals 30.

The information processor 11 and the cloud server 31 in the systems 1 and 2 may include an output unit. The output unit may be, for example, monitor displaying, printing, memory storage, or writing onto a storage medium.

The processor 112 in each of the systems 1, 2, and 3 determines the possibility of glaucoma based on the change rate in pulse wave amplitude (ΔPA) and the change rate in heart rate adjustment index (ΔCVRR). The processing unit 112 may perform determination using the change rate in pulse wave amplitude (ΔPA) as first index data. The processing unit 112 may also additionally use the change rate in heart rate adjustment index (ΔCVRR) as second index data.

Each processing of the image analysis unit, the first calculation unit, the second calculation unit, and the processor in each of the systems 1, 2, and 3 may be implemented when one or more micro processing units (MPUs), a central processing units (CPUs), or the like read and execute programs stored in the memory, or the image analysis unit, the first calculation unit, the second calculation unit, and the processing unit may be constituted of dedicated circuits or firmware to implement each processing.

The memory may include, for example, a random access memory (RAM), and a read only memory (ROM).

The mobile terminal 30 may include, for example, a smartphone, a tablet, a wearable terminal (including a smartwatch) or a VR device.

### EXAMPLE

Moving images of the palm of the subject are taken with the imaging analyzer in the first embodiment. The moving image before cold provocation and the moving image after cold provocation are taken. As a condition of the cold provocation, room temperature is set to 20° C. with an air conditioner, and a part of the subject beyond his or her right wrist is placed in a cold water at temperature of 4° C. for 1 minute. This is called cold water immersion, and cold water is an example of the coolant.

From the moving image data of 15 individuals in the healthy group before and after the cold provocation and the moving image data of 14 individuals in the glaucoma (NTG) group before and after the cold provocation, their respective pulse wave amplitudes and pulse wave intervals before and after the cold provocation are obtained.

With PA1 as a pulse wave amplitude just before the cold provocation, PA2 as a pulse wave amplitude after the lapse of a first prescribed time (for example, 4 minutes) from the time when application of the cold provocation is completed, and PA3 as a pulse wave amplitude after the lapse of a second prescribed time (for example, 6 minutes) from the time when the application of the cold provocation is completed, a change rate in pulse wave amplitude (ΔPA₂₋₁) at 4 minutes after the cold provocation with respect to just before the cold provocation, and a change rate in pulse wave amplitude (ΔPA₃₋₁) at 6 minutes after the cold provocation with respect to just before the cold provocation are obtained by the expression (1). Thus, the pulse wave amplitude after the lapse of the first prescribed time is used as PA2, and the pulse wave amplitude after the lapse of the second prescribed time is used as PA3, because in the glaucoma (NTG) group, the pulse wave does not show any change immediately after the application of the cold provocation and shows a change after a certain amount of time elapses. In FIG. 6, an average of the change rate in pulse wave amplitudes (ΔPA₂₋₁ and ΔPA₃₋₁) in the healthy group is shown by a dashed line and an average of the change rate in pulse wave amplitudes (ΔPA₂₋₁ and ΔPA₃₋₁) in the glaucoma (NTG) group is shown by a solid line. In FIG. 6, the horizontal axis represents elapsed time after the cold provocation, 0 minute indicates just before the cold provocation, and a change rate in pulse wave amplitudes (ΔPA₂₋₁ and ΔPA₃₋₁) at 4 minutes and 6 minutes after the cold provocation is plotted. Since the distinction between the healthy group and the glaucoma (NTG) group is clear at both 4 and 6 minutes, it is possible to classify the healthy group and the glaucoma (NTG) group by setting a middle value (e.g. 8.58) of the difference between an upper limit (e.g. 17%) and a lower limit (e.g. 0%) or a value close to the middle value (e.g. 7% to 9%) as the threshold.

In addition to a single threshold using the middle value, a plurality of thresholds can be set by dividing the difference between the upper limit and lower limit into three or four segments to allow classification into a pre-glaucoma group or a pseudo-glaucoma group. It is also possible to set thresholds by using a middle value of the difference between an inclination of the healthy group (dashed line) and an inclination of the glaucoma (NTG) group (solid line) or by dividing the difference into a plurality of segments.

In FIG. 6, a value that is larger than the lower limits of the healthy group at 4 minutes and 6 minutes after the cold provocation is set as the first threshold (Th₁), a value smaller than the upper limits in the NTG group is set as the third threshold (Th₂), and a middle value of these is set as the second threshold (Th₂) to allow classification into the healthy group, the pseudo-glaucoma group, the pre-glaucoma group, and the glaucoma (NTG) group.

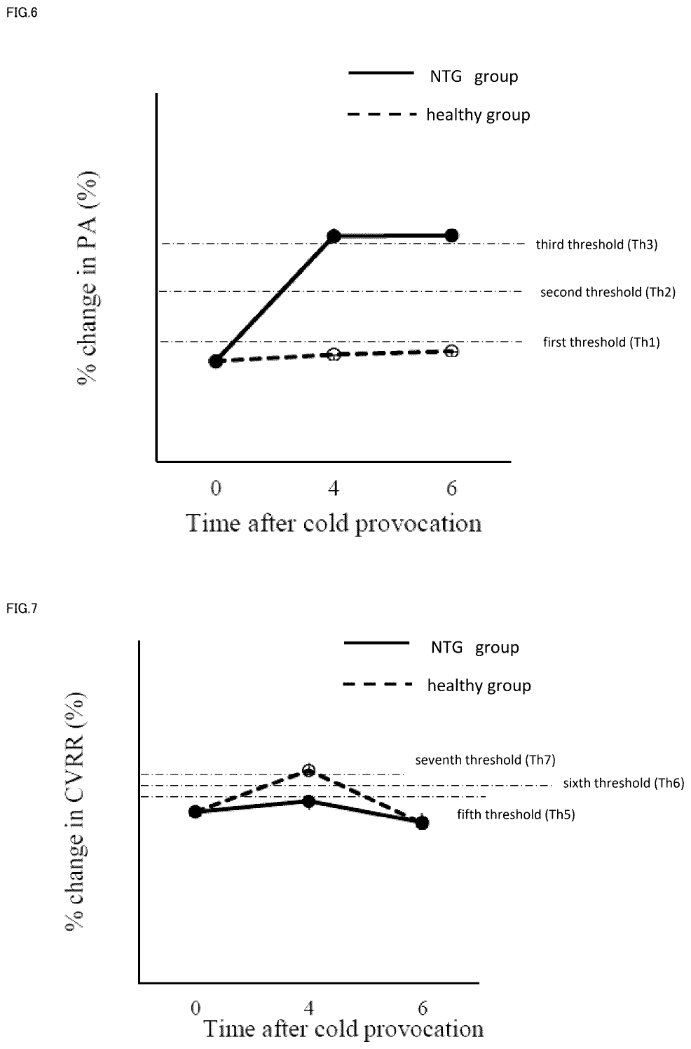

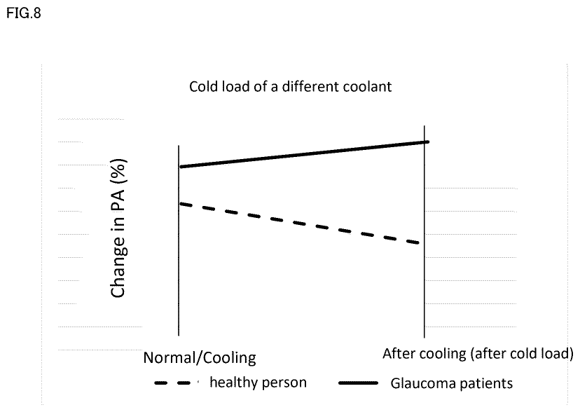

According to the expression (3), there are obtained a heart rate adjustment index CVPR1 just before the cold provocation, a heart rate adjustment index CVRR2 at the first prescribed time (e.g. 4 minutes) from the time when application of the cold provocation is completed, and the heart rate adjustment index CVRR3 after the lapse of the second prescribed time (e.g. 6 minutes) from the time when application of the cold provocation is completed. According to the expression (4), there are obtained a change rate in heart rate adjustment index (ΔCVRR₂₋₁) at 4 minutes after the cold provocation with respect to just before the cold provocation, and a change rate in heart rate adjustment index (ΔCVRR₃₋₁) at 6 minutes after the cold provocation with respect to just before the cold provocation. In FIG. 7, an average of the change rate in heart rate adjustment index (ΔCVRR₂₋₁ and ΔCVRR₂₋₁) in the healthy group is shown by a dashed line and an average in the NTG group is shown by a solid line. In FIG. 7, the horizontal axis represents elapsed time after the cold provocation, 0 minute indicates just before the cold provocation, and a change rate in pulse wave amplitudes (Δ PA₂₋₁ and ΔCVRR₃₋₁) at 4 minutes and 6 minutes after the cold provocation are plotted. Since the distinction between the healthy group and the NTG group is clear at 4 minutes, it is possible to classify the healthy group and the NTG group by setting a middle value (e.g. 5.5%) of the difference between an upper limit (e.g. 12%) and a lower limit (e.g. 1%) or a value close to the middle value (e.g. 5% to 6%) as the threshold.

In FIG. 7, a lower limit in the NTG group at 4 minutes after the cold provocation is set as the fifth threshold (Th₅), an upper limit in the healthy group is set as the seventh threshold (Th₇), and a middle value of these is set as the sixth threshold (Th₆) to allow classification into the healthy group, the pseudo-glaucoma group, the pre-glaucoma group, and the NTG group. The determination in the processor 112 is executed based on each set threshold. Each threshold is pre-stored in the memory.

As a coolant of another embodiment, a beverage can (for example, a coffee can) set to 5° C. was used. The change rate in pulse wave amplitude (ΔPA), under cold provocation in which the palm was cooled by the coolant, was evaluated. The pulse wave amplitude (PA1) was measured using the palm of each subject (in the healthy group and the glaucoma (NTG) group) in resting time. Each subject was then subjected to cold provocation by making each subject hold a can coffee at 5° C. for 2 minutes in the palm. Then, the pulse wave amplitude (PA2) was measured on the palm after the cold provocation. As a result, in the glaucoma (NTG) group, the change rate in pulse wave amplitude was above 100% and an increase after the cold provocation was recognized, whereas in the healthy group, the change rate in pulse wave amplitude was less than 100% and an increase after the cold provocation was not recognized. From the result, it was confirmed that even when the coolant was changed, the same result as in the case of cold water immersion was obtained and that the change in pulse wave amplitude peculiar to glaucoma (NTG) patients could be detected even in the case of cooling the palm only.

The determination using the change rate in pulse wave amplitude (ΔPA) is described with reference to FIG. 4. The following steps are implemented in any of the first to third embodiments.

In step S1, the image analysis unit obtains a pulse wave from a moving image just before the cold provocation and calculates the pulse wave amplitude PA1.

In step S2, the cold provocation is carried out. In step S3, the time of carrying out the cold provocation starts to be counted, and in step S4, it is determined whether 4 minutes has elapsed from the start of counting. The subject H or a third party may check with a timer. It is also possible to measure with a timer included in the imaging analyzer 10, the mobile terminal 20, and the wearable terminal 30. The same is true for step 6, which is described later.

In step S5, the image analysis unit obtains a pulse wave from a moving image after the lapse of 4 minutes, and calculates the pulse wave amplitude PA2.

In step S6, it is determined whether 6 minutes has elapsed from the start of counting.

In step S7, the image analysis unit obtains a pulse wave from a moving image after the lapse of 6 minutes, and calculates the pulse wave amplitude PA3.

In step S8, the first calculation unit calculates the change rate in pulse wave amplitude (ΔPA₂₋₁) at 4 minutes after the cold provocation with respect to just before the cold provocation, and the change rate in pulse wave amplitude (ΔPA₃₋₁) at 6 minutes after cold provocation with respect to just before the cold provocation.

In step S9-1, the processor determines whether or not each of the change rate in pulse wave amplitude (ΔPA₂₋₁) and the change rate in pulse wave amplitude (ΔPA₃₋₁) is smaller than the first threshold (Th₁). When the determination result is YES, the subject is determined to be in the healthy group in step S10-1.

When the determination result is NO, the processor determines in step S9-2 whether or not each of the change rate in pulse wave amplitude (ΔPA₂₋₁) and the change rate in pulse wave amplitude (ΔPA₃₋₁) is equal to or more than the first threshold (Th₁) and smaller than the second threshold (Th₂). When the determination result is YES, the subject is determined to be in the pre-glaucoma group in step S10-2.

When the determination result is NO, the processor determines in step S9-3 whether or not each of the change rate in pulse wave amplitude (ΔPA₂₋₁) and the change rate in pulse wave amplitude (ΔPA₃₋₁) is equal to or more than the second threshold (Th₂) and smaller than the third threshold (Th₃). When the determination result is YES, the subject is determined to be in the pseudo-glaucoma group in step S10-3.

When the determination result is NO, the processor determines in step S9-4 whether or not each of the change rate in pulse wave amplitude (ΔPA₂₋₁) and the change rate in pulse wave amplitude (ΔPA₃₋₁) is equal to or more than the third threshold (Th₃). When the determination result is YES, the subject is determined to be in the glaucoma (NTG) group in step S10-4. When the determination result is NO, no determination result available (for example, determination error) is determined in step S10-5.

In step S11, any one of the determination results in steps S10-1, S10-2, S10-3, S10-4, and S10-5 is transmitted or displayed.

In this determination flow, both the change rate in pulse wave amplitude (ΔPA₂₋₁) and the change rate in pulse wave amplitude (ΔPA₃₋₁) are used, though only one of these may be used for determination.

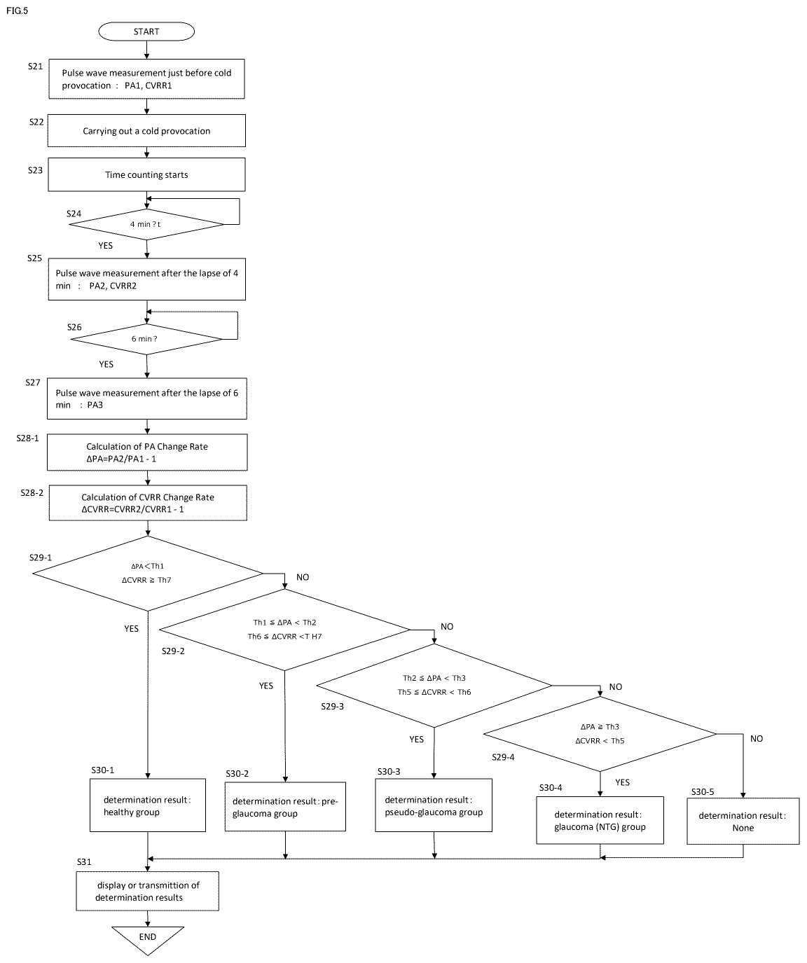

The determination using the change rate in pulse wave amplitude (ΔPA) and the change rate in heart rate adjustment index (ΔCVRR) is described with reference to FIG. 5. Following steps are also implemented in any of the first to third embodiments.

In step S21, the image analysis unit obtains a pulse wave and a pulse wave interval from a moving image just before the cold provocation, calculates the pulse wave amplitude PA1, and calculates the heart rate adjustment index CVRR₁.

In step S22, cold provocation is carried out. In step S23, counting of the time for carrying out the cold provocation is started, and in step 24, it is determined whether or not 4 minutes has elapsed from the start of counting. The subject H or a third party may check with a timer. It is also possible to measure with a timer included in the imaging analyzer 10, the mobile terminal 20, and the wearable terminal 30. The same is true for step 26, which is described later.

In step S25, the image analysis unit obtains a pulse wave and a pulse wave interval from the moving image after the lapse of 4 minutes, calculates the pulse wave amplitude PA2, and calculates the heart rate adjustment index CVRR2.

In step 26, it is determined whether or not 6 minutes has elapsed from the start of counting.

In step S27, the image analysis unit obtains a pulse wave from a moving image after the lapse of 6 minutes, and calculates the pulse wave amplitude PA3.

In step S28-1, the first calculation unit calculates the change rate in pulse wave amplitude (ΔPA₂₋₁) at 4 minutes after the cold provocation with respect to just before the cold provocation, and the change rate in pulse wave amplitude (ΔPA₃₋₁) at 6 minutes after the cold provocation with respect to just before the cold provocation.

In step S28-2, the second calculation unit calculates the change rate in heart rate adjustment index (ΔCVRR₂₋₁) at 4 minutes after the cold provocation with respect to just before the cold provocation.

In step S29-1, the processor determines whether or not each of the change rate in pulse wave amplitude (ΔPA₂₋₁) and the change rate in pulse wave amplitude (ΔPA₃₋₁) is smaller than the first threshold (Th₁). The processor then determines whether or not the change rate in heart rate adjustment index (ΔCVRR₂₋₁) is equal to or more than the seventh threshold (Th₇). When both the determination results are YES, the subject is determined to be in the healthy group in step S30-1.

When the determination result is NO, the processor determines in step S29-2 whether or not each of the change rate in pulse wave amplitude (ΔPA₂₋₁) and the change rate in pulse wave amplitude (ΔPA₃₋₁) are equal to or more than the first threshold (Th₁) and smaller than the second threshold (Th₂). The processor then determines whether or not the change rate in heart rate adjustment index (ΔCVRR₂₋₁) is equal to or more than the sixth threshold (Th₆) and is smaller than the seventh threshold (Th₇). When both the determination results are YES, the subject is determined to be in the pre-glaucoma group in step S30-2.

When the determination result is NO, the processor determines in step S29-3 whether or not each of the change rate in pulse wave amplitude (ΔPA₂₋₁) and the change rate in pulse wave amplitude (ΔPA₃₋₁) are equal to or more than the second threshold (Th₂) and smaller than the third threshold (Th₃). The processor then determines whether or not the change rate in heart rate adjustment index (ΔCVRR₂₋₁) is equal to or more than the fifth threshold (Th₅) and is smaller than the sixth threshold (Th₂). When both the determination results are YES, the subject is determined to be in the pseudo-glaucoma group in step S30-3.

When the determination result is NO, the processor determines in step S29-4 whether or not each of the change rate in pulse wave amplitude (ΔPA₂₋₁) and the change rate in pulse wave amplitude (ΔPA₃₋₁) is equal to or more than the third threshold (Th₃). The processor then determines whether or not the change rate in heart rate adjustment index (ΔCVRR₂₋₁) is smaller than the fifth threshold (Th₅). When both the determination results are YES, the subject is determined to be in the glaucoma group (NTG group) in step S30-4. When the determination result is NO, no determination result available (for example, determination error) is determined.

In step S31, any one of the determination results in steps S30-1, S30-2, S30-3, S30-4, and S30-5 is transmitted or displayed.

In this determination flow, both the change rate in pulse wave amplitude (ΔPA₂₋₁) and the change rate in pulse wave amplitude (ΔPA₃₋₁) are used, though only one of these may be used for determination.

The accuracy of determination is considered to be enhanced when the processor performs determination using the change rate in pulse wave amplitude (ΔPA) first and then carries out determination using the change rate in heart rate adjustment index (ΔCVRR). Although the determination using the pulse amplitude change rate (ΔPA) is to be performed first as described in the foregoing, the possibility and degree of glaucoma may be determined by using only the change rate in heart rate adjustment index (ΔCVRR).

In the above example, the change rate in pulse wave amplitude (ΔPA) and the change rate in heart rate adjustment index (ΔCVRR) are calculated based on data measured at the same time. They may also be calculated using separately measured data.

### (Glaucoma Determination Method)

A glaucoma determination method, for determining a possibility of glaucoma and/or a degree of glaucoma, executed by a computer, includes: an analysis step of obtaining a pulse wave of blood flow of a subject from a moving image or reflected light and calculating a pulse wave amplitude and a pulse wave interval; a first calculation step of calculating a change rate in pulse wave amplitude (ΔPA) that is a change rate in the pulse wave amplitude during prescribed time; a second calculation step of calculating a change rate in heart rate adjustment index (ΔCVRR); and a processing step of determining, based on the change rate in pulse wave amplitude (ΔPA) and the change rate in heart rate adjustment index (ΔCVRR), the possibility of glaucoma and/or the degree of glaucoma of the subject, and outputting information related to the possibility of glaucoma and/or the degree of glaucoma.

### (Glaucoma Determination Program)

A program (1) for causing a computer to execute, based on index data related to blood flow of a subject, the processes of: obtaining a pulse wave of blood flow of a subject from a moving image or reflected light and calculating a pulse wave amplitude and a pulse wave interval; calculating a change rate in pulse wave amplitude (ΔPA) that is a change rate in the pulse wave amplitude during prescribed time; calculating a change rate in heart rate adjustment index (ΔCVRR); determining, based on the change rate in pulse wave amplitude (ΔPA) and the change rate in heart rate adjustment index (ΔCVRR), a possibility of glaucoma and/or a degree of glaucoma of the subject; and outputting information related to the possibility of glaucoma and/or the degree of glaucoma.

Another program (2) causes execution of the processes of: receiving a change rate in pulse wave amplitude (ΔPA) and a change rate in heart rate adjustment index (ΔCVRR); determining a possibility of glaucoma and/or a degree of glaucoma of the subject; and outputting information related to the possibility of glaucoma and/or the degree of glaucoma.

The programs (1) and (2) may be stored in a memory of the glaucoma determination system, and be read from the memory and executed as required.

### Other Embodiments

(1) In the first embodiment, the imaging analyzer 10 analyzes a moving image and obtains the pulse wave amplitude and the pulse wave interval. Without being limited to this, the moving image may be sent to the information processor 11 and similar processing may be carried out in the information processor 11.

(2) The information processor 11 in the first embodiment and the second embodiment may be constituted of a cloud server.

(3) The mobile terminal 20 in the second embodiment may have an application of the glaucoma determination program (1) or (2) installed thereon so that the mobile terminal 20 may execute the function of the information processor 11. The mobile terminal 20 may send the determination result (which may or may not include terminal identification information) to the information processor 11, and the information processor 11 may store the determination result in its memory or database.

(4) The wearable terminals 30 in the third embodiment may each have an application of the glaucoma determination program installed thereon, so that each of the wearable terminals 30 may execute the function of the cloud server 31. The wearable terminals 30 may each send the determination result (which may or may not include the terminal identification information) to the cloud server 31, and the cloud server 31 may store the determination result in its memory or database.

(5) In the above example, a change in the amount of blood flow obtained from before and after cold provocation applied to the hand is used for determination. However, the present invention is not limited thereto, and the region of the blood flow may be the forehead, cheek, neck, or the like.

(6) The load is not limited to cold provocation and other kinds of load may be used.

### REFERENCE SIGNS LIST

1, 2, 3 Glaucoma determination system
10 Imaging analyzer
11 Information processor
20 Mobile terminal
30 Wearable terminal
31 Cloud server
111 Reception unit
112 processor
113 Transmission unit
114 Image analysis unit
115 First calculation unit
116 Second calculation unit

1. A glaucoma determination system, comprising a processor that acquires index data related to blood flow of a subject, determines, based on the acquired index data, a possibility of glaucoma in an eye of the subject, and outputs information related to the determined possibility of glaucoma.

2. The glaucoma determination system according to claim 1, wherein the processor determines and outputs, based on the index data, the possibility of glaucoma of the subject as well as a degree of glaucoma of the subject.

3. The glaucoma determination system according to claim 1, wherein the index data is a change rate in pulse wave amplitude that is a change rate in pulse wave amplitude during a prescribed time.

4. The glaucoma determination system according to claim 3, wherein the index data includes fluctuation of a pulse wave interval.

5. The glaucoma determination system according to claim 1, wherein the processor determines the possibility of glaucoma of the subject based on a comparison result between the index data and a prescribed threshold.

6. The glaucoma determination system according to claim 2, wherein the processor determines the degree of glaucoma of the subject based on a comparison result between the index data and a prescribed threshold.

7. The glaucoma determination system according to claim 3, further comprising a first calculation unit that calculates the change rate in pulse wave amplitude.

8. The glaucoma determination system according to claim 4, further comprising a second calculation unit that calculates the fluctuation of the pulse wave interval from the blood flow.

9. The glaucoma determination system according to claim 1, wherein the blood flow of the subject is blood flow at a specific region.

10. The glaucoma determination system according to claim 1, wherein the index data is based on data related to the blood flow of the subject after a prescribed load is applied to the subject, the prescribed load being large enough to change the blood flow of a person who may have glaucoma.

11. The glaucoma determination system according to claim 10, wherein the index data determines whether or not a prescribed time elapses since the prescribed load is applied to the subject, and is calculated based on post-load application data that relates to the blood flow of the subject measured during a period from application of the prescribed load on the subject to the lapse of the prescribed time.

12. The glaucoma determination system according to claim 11, wherein the index data is calculated based on a comparison result between pre-load application data that pertains to the blood flow of the subject measured prior to the application of the prescribed load on the subject and the post-load application data.

13. The glaucoma determination system according to claim 10, wherein the prescribed load is applied to the subject by cooling the subject with a prescribed coolant.

14. The glaucoma determination system according to claim 1, comprising:
at least one of an imaging device, a wearable device, and an information processing terminal for acquiring the index data related to the blood flow of the subject; and
at least one of a computer and a cloud server having the processing unit.

15. The glaucoma determination system according to claim 1, comprising at least one of an imaging device, a wearable device, and an information processing terminal for acquiring the index data related to the blood flow of the subject, wherein the at least one device includes the processor.

16. A glaucoma determination method to be executed by a computer, the method comprising the processing steps of:
acquiring index data related to blood flow of a subject; and
determining, based on the index data related to the blood flow of the subject, a possibility of glaucoma in an eye of the subject, and outputting information related to the possibility of the glaucoma.

17. A program causing a computer to execute the processes of:
acquiring index data related to blood flow of a subject;
determining, based on the index data related to the blood flow of the subject, a possibility of glaucoma in an eye of the subject; and
outputting information related to the possibility of the glaucoma.
